(19) United States
(12) Patent Application Publication
(10) Pub. No.: US 2011/0150960 A1
(43) Pub. Date: Jun. 23, 2011

(54) HAZARDOUS SUBSTANCE REMOVING MATERIAL AND METHOD FOR REMOVING HAZARDOUS SUBSTANCE

(76) Inventor: Hiroshi Iwanaga, Kanagawa (JP)

(21) Appl. No.: 12/737,611

(22) PCT Filed: Jul. 29, 2009
(86) PCT No.: PCT/JP2009/063831

§ 371 Date: Jan. 28, 2011

(51) Int. Cl. A01N 25/00; C07K 17/00; A01N 37/18; A01P 1/00; B82Y 5/00

(52) U.S. Cl. 424/411; 530/391.1; 424/178.1; 977/755; 977/902

(57) ABSTRACT

It is an object of the present invention to provide a hazardous substance removing material, which efficiently captures hazardous substances derived from microorganisms such as bacteria or viruses and rapidly inactivates them, so as to minimize the their influences on human bodies, and which is able to allow an antibody to be supported on a carrier by a simple method, and which has an improved antibody use efficiency. The present invention provides a hazardous substance removing material consisting of a carrier on which an antibody and a sugar chain affinity substance having an affinity for a sugar chain in the Fc region of the antibody are supported.

### TECHNICAL FIELD

The present invention relates to a hazardous substance removing material capable of selectively inactivating bacteria or viruses and a method for removing a hazardous substance using the same.

### BACKGROUND ART

In recent years, infectious diseases caused by bacteria, molds, viruses, and the like have been recognized as social problems. For instance, there is a concern of mass infection in general public places such as hospitals and public facilities. Particularly in the case of hospital infection, the misuse of antibiotics and the like causes the generation of MRSA (Methicillin-resistant Staphylococcus aureus), for example.

In view of the above, recent buildings are provided with a duct in each room in such a manner that air is circulated through the duct, using an air-conditioner, so as to control the room temperature or other conditions of the whole building. Thus, bacteria, molds, viruses, and the like floating in a facility are often diffused into the entire facility through such an air conditioner. Therefore, it is considered that blocking of a route for air-mediated infection is particularly effective. Specifically, a finely woven filter is provided to an air distribution part of an air conditioner, air purifier, or the like, such that bacteria, mold, viruses, or media therefore such as floating fine objects (e.g., dusts) in air are allowed to be adsorbed to the filter. Alternatively, titanium oxide or a strongly acidic sterilizing zone is provided to the same such that bacteria, molds, and viruses passing therethrough are inactivated and removed.

However, upon the removal by adsorption, if a hazardous substance is a bacterium, virus, or the like, bacteria having captured with a filter might be detached therefrom so as to be reactivated and affect human bodies. In addition, in the case of a method for inactivating a hazardous substance by allowing the hazardous substance to pass through titanium oxide or a strongly acidic sterilizing zone so as to inactivate the hazardous substance, inactivation is time-consuming to a certain extent and the effects obtained thereby are not always sufficient, which has been problematic.

Japanese Patent No. 3642340 describes a method for removing a hazardous substance in a gas phase atmosphere, using a hazardous substance removing substance in which an antibody is supported on a carrier; which is characterized in that it comprises controlling humidity in the atmosphere around the aforementioned antibody so that the antibody exhibits activity at the humidity. Japanese Patent No. 3642340 also describes that the Fc portion of the antibody is allowed to bind to the carrier, so that Fab that captures a hazardous substance becomes outward against the carrier and the contact probability of Fab with such hazardous substance increases, and thereby the hazardous substance can be efficiently captured. However, even in the method of Japanese Patent No. 3642340, antibody use efficiency is low. Thus, in order to more efficiently use the Fab portion, it has been necessary to carry out a special step for the antibody or the carrier. Hence, this method has been problematic in terms of low productivity.

On the other hand, International Publication WO2005/35586 describes a pharmaceutical composition which comprises a fusion protein molecule of a binding protein and an antibody Fc region having an N-glycoside-binding complex sugar chain. However, International Publication WO2005/35586 does not relate to an antibody-supported hazardous substance removing material.

### DISCLOSURE OF THE INVENTION

It is an object of the present invention to solve the problems of the conventional hazardous substance removing materials. Namely, it is an object of the present invention to provide a hazardous substance removing material, which efficiently captures hazardous substances derived from microorganisms such as bacteria or viruses and rapidly inactivates them, so as to minimize the their influences on human bodies, and which is able to allow an antibody to be supported on a carrier by a simple method, and which has an improved antibody use efficiency. Moreover, it is another object of the present invention to provide a method for efficiently removing a hazardous substance using the aforementioned hazardous substance removing material.

As a result of intensive studies directed towards achieving the aforementioned objects, the present inventor has found that a hazardous substance removing material, which efficiently captures hazardous substances and rapidly inactivates them, so as to minimize their influences on human bodies, and which is able to allow an antibody to be supported on a carrier by a simple method, and which has an improved antibody use efficiency, can be obtained by allowing an antibody and a sugar chain affinity substance having an affinity for a sugar chain in the Fc region of the antibody to be supported on a carrier, thereby completing the present invention.

The present invention provides a hazardous substance removing material consisting of a carrier on which an antibody and a sugar chain affinity substance having an affinity for a sugar chain in the Fc region of the antibody are supported.

Preferably, the antibody is IgG.

Preferably, the sugar chain affinity substance has the same oligosaccharide unit as that of a sugar chain in the Fc region of IgG

Preferably, the sugar chain affinity substance is a sugar chain containing at least one type selected from among glucose, galactose, mannose, xylose, fucose, N-acetylglucosamine, N-acetylgalactosamine, and N-acetylneuraminic acid.

Preferably, the surface of the carrier is coated with the sugar chain affinity substance.

Preferably, the hazardous substance removing material of the present invention has a hydrophilic polymer as well as the sugar chain affinity substance on the carrier.

Preferably, the hydrophilic polymer has at least one type of functional group selected from among a hydroxyl group, an amino group, an amide group, a carboxylic acid group, and a quaternary amino group.

Preferably, a layer comprising the sugar chain affinity substance has an average thickness of 5 to 20 nm.

Preferably, the antibody is derived from ostriches.

The present invention further provides a method for removing hazardous substance, which comprises removing a hazardous substance from a gas phase or a liquid phase using the aforementioned hazardous substance removing material of the present invention.

According to the present invention, by allowing an antibody and a sugar chain affinity substance having an affinity for a sugar chain in the Fc region of the antibody to be supported on a carrier, the effective amount of the supported antibody can be increased, and thus, hazardous substances can be reliably inactivated with a small amount of antibody. Furthermore, according to the present invention, it has become possible to provide a hazardous substance removing material having an improved preservative quality as well as an improved rate of capturing airborne bacteria. According to the method of the present invention, an air purifier or a liquid purifier capable of efficiently removing hazardous substances in a gas phase or a liquid phase can be produced, which is thus very useful in the industry.

### PREFERRED EMBODIMENT OF THE INVENTION

Hereinafter, the present invention will be described more in detail.

The hazardous substance removing material of the present invention is characterized in that it consists of a carrier on which an antibody and a sugar chain affinity substance having an affinity for a sugar chain in the Fc region of the antibody are supported.

### (1) Carrier

A main material which forms a carrier used in the present invention is preferably a fiber comprising, as a main component, at least one selected from the group consisting of cellulose ester, vinylon, acrylic, and polyurethane. In addition, as a main material which forms a carrier, a fiber comprising, as a main component, polyamide is also preferable. According to the present invention, the term “main component” means a component that accounts for 25% or more in terms of mass fraction with respect to the total mass of fibers.

According to the present invention, the term “cellulose ester” refers to a cellulose derivative obtained by esterifying a hydroxyl group of cellulose with an organic acid. Examples of an organic acid used for esterification include fatty carboxylic acids such as acetic acid, propionic acid, and butyric acid and aromatic carboxylic acids such as benzoic acid and salicylic acid. They may be used alone or in combination. The rate of substitution of a hydroxyl group of cellulose with an ester group is not particularly limited; however, it is preferably 60% or more.

According to the present invention, a cellulose acylate fiber is preferable among the group of main materials which form a carrier. The term “cellulose acylate” used herein refers to cellulose ester in which some or all of hydrogen atoms of a hydroxyl group of cellulose are substituted with an acyl group. Examples of an acyl group include an acetyl group, a propionyl group, and a butylyl group. In terms of structure, a single group among the above examples may be substituted, or two or more acyl groups may be subjected to mixed substitution. The total sum of degrees of acyl group substitution is preferably 2.0 to 3.0, more preferably 2.1 to 2.8, and particularly preferably 2.2 to 2.7. Among them, cellulose acetate, cellulose acetate propionate, or cellulose acetate butylate capable of achieving such degree of substitution is preferable, and cellulose acetate is most preferable. In general, it has been known that a solvent for cellulose acylate varies depending on the degree of esterification. It is also possible to produce a carrier with cellulose acylate having a high esterification rate in advance and then subject the carrier to alkali hydrolysis treatment or the like for hydrophilicization of the surface thereof.

It is possible to form a sufficiently practical hazardous substance removing material consisting of a cellulose acylate fiber. However, in order to further improve strength, dimensional stability, and the like, a carrier may be formed with a mixed fiber (e.g., polyester-based fiber/polyolefin-based fiber/polyamide-based fiber/acrylic-based fiber). When a mixed fiber is used, the mass fraction of a cellulose acylate fiber is preferably 50% or more and more preferably 70% or more.

According to the present invention, a polyamid fiber is preferable among the group of main materials which constitutes a carrier.

According to the present invention, the term “polyamide” refers to a fiber comprising a linear polymer having a chemical structure unit comprising an amide bond.

Among polyamides, a linear aliphatic polyamide, which is a combination of an aliphatic diamine such as ethylenediamine, 1-methylethylenediamine, 1,3-propylenediamine, or hexamethylenediamine and an aliphatic dicarboxylic acid such as malonic acid, succinic acid, or adipic acid, is preferable. Nylon 66 is particularly preferable.

In addition to the above diamine and dicarboxylic acid, aliphatic polyamide comprising a single component or copolymer components selected from among the following examples can be used: lactams such as ε-caprolactam and laurolactam; aminocarboxylic acids such as aminocaproic acid and aminoundecanoicacid; and para-aminomethyl benzoic acid. Nylon 6 produced using ε-caprolactam alone is particularly preferable.

In addition to the above, the following may be used: an aliphatic polyamide in which cycloaliphatic diamine such as cyclohexanediamine, 1,3-bis(aminomethyl)cyclohexane, or 1,4-bis(aminomethyl)cyclohexane is partially or entirely used as a material aliphatic diamine; and/or an aliphatic polyamide in which cycloaliphatic dicarboxylic acid such as 1,4-cyclohexane dicarboxylic acid, hexahydroterephthalic acid, or hexahydroisophthalic acid is partially or entirely used as dicarboxylic acid.

Further, examples of the above polyamide further include a polyamide with decreased water absorbability and an improved elastic modulus in which aromatic diamine such as aliphatic paraxylylene diamine (PXDA) or metaxylylene diamine (MXDA) and aromatic dicarboxylic acid such as terephthalic acid are partially used as starting materials. Moreover, a polymer having a side chain comprising an amide bond such as polyacrylic acid amide, poly(N-methylacrylic acid amide), or poly(N,N-dimethylacrylic acid amide) may be used.

Among polyamides, nylon 66 or nylon 6 is most preferable. This is because the following properties of such polyamide are preferable to be used as the carrier of the present invention: appropriate hygroscopic properties derived from amide bonds; ease of inducing fiber axis orientation of a molecular chain comprising a long-chain fatty acid having an appropriate length that results in relatively high extensibility; a dynamic and kinetic tendency to not be melted due to high melting temperature and thermal capacity (resistance to melting); flexibility of a molecular chain comprising a long-chain fatty acid; and a tendency to not cause fibrillation or kink band formation (such tendency being imparted as a result of formation of a hydrogen bond between amide bonds), that is to say, repetitive bending and stretching properties.

Preferably, a polyamide in which an amide bond in a chemical structure unit exists on a side chain but not on a main chain can be used. Examples thereof include polyacrylamides such as poly(N-isopropylacrylamide), poly(N,N-dimethylacrylamide), and poly(N-hexylacrylamide). In general, a polymer having a side chain comprising an amide bond has high hydrophilicity and thus tends to be swollen/deformed. Thus, it is preferable that a physically crosslinked polymer be formed with the use of a gelatinization phenomenon or a polymer be hydrophobized by a method comprising introducing an alkyl group, for example.

Likewise, in order to improve strength or dimensional stability, a carrier may be reinforced with other appropriate structural materials such as metals, high-molecular materials, and ceramics. It is desirable that such reinforcing materials be used for a part which is not positioned on the substantially outermost surface of a face to which a hazardous substance removing material is applied (such material being used for, for example, the face located opposite to such face or a core material).

According to the present invention, the term “vinylon” refers to a fiber comprising a linear polymer containing vinyl alcohol units (65% by mass or more) and having a moisture regain of less than 7% obtained at least 1 week after placement of such fiber in an environment at a temperature of 20° C. and at a humidity of 65%. Such fiber may be obtained by formalizing a hydroxyl group of vinyl alcohol. Also, it may be a polymer obtained by subjecting a hydroxyl group to boric acid crosslinking or a non-formalized fiber subjected to a waterproof treatment by a known method such as an alkaline spinning method or a cooled gel spinning method. The above fiber may contain, as non-vinyl-alcohol-unit component, an ethylene chain or a vinyl acetate chain. However, it is preferably a fiber formed with a vinyl alcohol carrier. Further, it is most preferably a non-formalized fiber obtained by cooled gel spinning. This is because a non-formalized fiber has uniform properties and high degree of orientation/crystallization and thus excellent mechanical properties and reliability can be obtained.

In general, vinylon is superior to other fibers in terms of high strength, high elastic modulus, appropriate hydrophilicity, weather resistance, chemical resistance, adhesiveness, and the like. Thus, the preferable properties thereof can be used for the carrier of the present invention.

According to the present invention, the term “acrylic” refers to a fiber comprising recurring units of an acrylonitrile group (mass percentage: 40% or more). Examples thereof include a homopolymer of acrylonitrile; a copolymer of acrylnitrile and a nonionic monomer such as acrylic ester, methacrylic ester, or vinyl acetate; a copolymer of acrylonitrile and an anionic monomer such as vinylbenzenesulfonate or allylsulfonate; and a copolymer of acrylonitrile and a cationic monomer such as vinylpyridine or methylvinylpyridine. A promix fiber which is formed from acrylonitrile and milk casein is included in this category.

In general, an acrylic fiber is produced by an organic solvent wet spinning method. In this method, when a spinning stock solution is formed into a coagulated thread in a coagulating bath, water serving as a coagulant is mixed with the spinning stock solution that is spinning-twisted from a nozzle and a spinning solvent is externally diffused from the spinning-twisted stock solution. At such time, water and an organic solvent (e.g., DMF or DMAc) are mutually diffused such that a polymer deposits, resulting in the formation of a line of coagulated thread having a structure in which many cavities are connected to each other in a net form. In addition, such thread is characterized by deformation of a fiber section caused by volume contraction as a result of diffusion of a solvent into a coagulating bath during coagulation and by formation of concave-convex portions as a result of macrofibril structure formation on the surface thereof. Such fine structure is preferable as a structure of a carrier used in the present invention in terms of an increase in specific surface area or the ease of antibody loading.

An acrylic fiber used in the present invention varies depending on the composition of a starting material polymer, a spinning method, post-treatment conditions during production, and the like. However, in general, a bulky fiber having appropriate hydrophilicity and high weather resistance can be obtained, which is advantageous.

The term “polyurethane” used in the present invention refers to a fiber comprising a linear synthetic polymer in which bonds between monomers or basic substrate polymer units are mainly urethane bonds. Preferably such fiber contains a polyurethane segment at a mass percentage of 85% or more. Preferably, such polyurethane is a block copolymer of segmented polyurethane comprising a soft segment that is soft and have a molecular weight of several thousands and a low melting point and a hard segment that is rigid and have high cohesion and a high melting point. For a soft segment, polyether such as polypropylene glycol or polytetramethylene glycol can be used. For a hard segment, a urethane group formed with 4,4′-diphenylmethane diisocyanate, m-xylene diisocyanate, or the like can be used. Polyurethane is generally characterized by a high elasticity. Also, it is further characterized by good extensibility, high restoring force upon expansion and contraction, antidegradation properties better than those of rubber materials, formation into thin fibers, and the like, although the characteristics thereof vary depending upon differences in terms of a primary structure of a high-molecular chain such as the distribution and chemical structure of each segment and upon differences in terms of a secondary structure derived from different spinning conditions. Thus, when polyurethane is used as a carrier of the present invention, such characteristics can be utilized.

In addition to the aforementioned carrier, there can be used various types of carriers including hydrophobic fibers such as polyolefin and polyester, which should be subjected to gas phase surface modification treatments such as an oxygen plasma treatment or a UV/ozone treatment, chemical modification treatments using a compound having a hydrophilic group, or hydrophilic surface treatments involving coating with a hydrophilic polymer.

Regarding mechanical and physical properties and dimensional stability of a fiber constituting a carrier, the tensile elastic modulus in a dried state is preferably 25% or more. The term “tensile elastic modulus in a dried state” used herein refers to the degree of elongation at break of a fiber in a tensile test at 20° C., provided that such fiber has been dried for a sufficiently long period of time. In general, a fiber having a tensile elastic modulus in a dried state of 10% or more is preferable for processing such as fabric formation. In order to prevent breakage upon filter processing or practical use (such breakage leading to reduction in filtration efficiency), the tensile elastic modulus is preferably 25% or more, more preferably 30% or more, and most preferably 35% or more.

The official moisture regain of the fiber constituting the carrier is preferably not less than 1.0% to less than 7%, more preferably not less than 3.0% to less than 6.5%, most preferably not less than 5.0% to less than 6.5%. Within the above range of official moisture regain, the expression of the activity of a supported antibody and the mechanical strength, rigidity, dimensional change stability in a use environment (particularly humidity) of a carrier can be achieved. Further, a filter obtained therewith can exhibit high performance and reliability.

In addition, the term “moisture regain” used herein refers to an official moisture regain. The term “official moisture regain” refers to a moisture regain of a fiber that has been left in an environment at 20° C. and at a relative humidity of 65% for long period of time. Moreover, when a fiber is a mixed fiber further containing a different fiber, the term refers to the official moisture regain of the total mixed fibers.

Preferably, the surface of a fiber constituting a carrier has fine concave-convex portions several tens nanometers to several micrometers in size. The shape of a concave-convex portion may be a three-dimensionally shaped groove or ridge which is formed in the direction parallel to the fiber direction or in the direction vertical to the same, that is to say, in a concentric direction with respect to the fiber axis. Such three-dimensionally shaped groove or ridge may exist at an arbitrary proportion or density, provided that an arbitrary angle is formed between such groove or ridge and a line extending in the direction parallel thereto, in the direction vertical thereto, or in the direction between such parallel direction and such vertical direction. A sample obtained by a known method for cellulose acetate fiber spinning is known to have a fiber section having a variable chrysanthemum-like shape as a result of skin layer formation on the surface thereof and depression of a skin layer due to solvent drying. In a preferred embodiment, such concave-convex portions are used for the present invention.

The above fine concave-convex portions several tens nanometers to several micrometers in size may have holes and/or projections. Preferably, such holes or projections have an average diameter of 50 nm to 1 μm. Such holes and projections can be formed by, for example, cavitation of a solution or they can be formed in a spinning step of a method using a solution in which a fine dispersoid is dispersed (e.g., a mixture containing a slurry in which barium sulfate particles are dispersed) or in a subsequent step by a method involving hydrolysis of an acyl group, surface oxidation treatment, or the like (e.g., the exposure of a cellulose portion on the fiber surface with the use of an alkaline water solution followed by generation of microcraters by an enzyme treatment).

The average fiber diameter of a fiber used for the hazardous substance removing material of the present invention is preferably 50 μm or less, more preferably 10 μm or less, particularly preferably 1 μm or less, and most preferably 100 nm or less. The average fiber diameter of the present invention is obtained by measuring the diameters of fibers in arbitrarily selected 300 sites on a scanning electron microscope (SEM) image for observation and averaging the results by calculation.

As to the method for producing the fiber used in the present invention, there are typical production methods such as melting spinning, wet spinning, dry spinning, and dry-wet spinning, and methods in which the fiber is made fine by a physical process (such as strong mechanical shearing using an ultrahigh pressure homogenizer), although dry spinning or dry-wet spinning is preferably employed for obtaining a stable quarity of product. For producing an uniform fiber having an average fiber diameter of 100 nm or less, the electrospinning method disclosed in “Kakou Gijyutsu (Processing Technology)”, 2005, Vol. 40, No. 2, p. 101 and p. 167; “Polymer International”, 1995, Vol. 36, pp. 195-201; “Polymer Preprints”, 2000, Vol. 41(2), p. 1193; “Journal of Macromolecular Science: Physics”, 1997, B36, p. 169; and the like is preferably used.

Regarding the solvent used for the spinning, any solvent may be used as long as it dissolves the resin used for synthetic resin fibers. Examples thereof include: chloride-based solvents such as methylene chloride, chloroform, and dichloroethane; amide-based solvents such as dimethylformamide, dimethylacetamide, and N-methylpyrrolidone; ketone-based solvents such as acetone, ethyl methyl ketone, methyl isopropyl ketone, and cyclohexanone; ether-based solvents such as THF and diethyl ether; and alcohol-based solvents such as methanol, ethanol, and isopropanol. These solvents may be used either singularly, or in mixtures of a plurality of types thereof.

The resin solution used for the electrospinning method may be added with a salt such as lithium chloride, lithium bromide, potassium chloride, and sodium chloride.

Preferably, fibers constituting a carrier of the hazardous substance removing material of the present invention partially adhere to each other such that a structure forming a three-dimensional network is obtained. The use of such structure results in the improvement of mechanical tolerance upon processing or practical use, leading to the improvement of reliability of the hazardous substance removing material. Further, antibody-supporting properties of the present invention can be improved. Adhesion between fibers can be observed by a method involving SEM or the like. The density of fiber adhesion points is preferably 10 adhesion points or more in a 1-mm square on the projected surface area of the hazardous substance removing material and preferably 100 adhesion points or more in the same.

Regarding a method for forming adhesion points, adhesion points may be formed by a dry spinning method or by a melt spinning method. After spinning, adhesion point formation treatment may be carried out by heating or adding an adhesive/plasticizing solvent or the like. In view of production cost, it is preferable to form adhesion points by a dry spinning method with the use of an appropriate solution formulation.

### (2) Sugar Chain Affinity Substance Having Affinity for Sugar Chain in Fc Region of Antibody

In the hazardous substance removing material of the present invention, a sugar chain affinity substance which has an affinity for a sugar chain in the Fc region of an antibody is supported on a carrier.

Antibody is a biological polymer that acts in the immunomechanism of a living body. There are 5 types of mammalian antibodies, namely, IgG, IgE, IgD, IgM, and IgA. An antibody corresponding to IgG contained in the yolk of Ayes is referred to as IgY. In general, the aforementioned antibodies have a sugar chain. Any type of antibody may be used herein. As the antibody used in the present invention, IgG and IgY are preferable, in that they are supported on a substrate, and in that the concentrations thereof in serum or yolk are high and they are easily used. IgG is particularly preferable.

The IgG antibody has a chain consisting of more than a dozen of monosaccharides connected with one another, which is referred to as a sugar chain. In human IgG for example, such sugar chain binds to aspartic acid (Asn297) that is the amino acid at position 297 from the N-terminus of an H chain. In recent years, studies regarding the structure or functions of this sugar chain have progressed, and as a result, it has been found that this sugar chain plays an important role for expression of the function of the antibody. The sugar chain affinity substance used in the present invention preferably has the same oligosaccharide unit as that of a sugar chain in the Fc region of IgG.

The term “sugar chain” is used in the present invention to mean a group of compounds, in which various types of sugars bind to one another via a glycoside bond. The number of such binding sugars varies from two to several tens of thousands. A group of approximately 10 sugars is referred to as an oligosaccharide. Examples of the simplest sugar chains are amylose and cellulose, in which numerous a glucose molecules linearly bind to one another.

As a sugar chain affinity substance used in the present invention, a sugar chain, in which units containing at least one type selected from among glucose, galactose, mannose, xylose, fucose, N-acetylglucosamine, N-acetylgalactosamine, and N-acetylneuraminic acid, bind in a chain form, is preferable. It is particularly preferable that the contents of N-acetylglucosamine and N-acetylneuraminic acid that constitute the terminus of the sugar chain and fucose as a side chain be high (which causes a high efficiency and a high stabilization effect).

The additive amount of the sugar chain affinity substance of the present invention is preferably 0.1% by mass to 1,000% by mass, more preferably 1% by mass to 500% by mass, and most preferably 5% by mass to 200% by mass, with respect to the mass of the antibody. When the additive amount of the present sugar chain affinity substance is within the aforementioned range, the present sugar chain affinity substance does not undergo significant function suppression due to coating on the antigen-recognizing site of an antibody or steric hindrance occurring during the reaction with an antigen, and as a result, the effect of the present invention can be exhibited.

The surface of the carrier is preferably coated with the sugar chain affinity substance. The average thickness of a layer consisting of the sugar chain affinity substance is preferably between 5 and 20 nm.

The sugar chain affinity substance not only enables effective utilization of an antigen-recognizing site, but it can also exhibit functions such as provision of a hydrophilic site and antibody protein-stabilizing action.

### (3) Hydrophilic Polymer

In the present invention, a hydrophilic polymer as well as a sugar chain affinity substance can be supported on a carrier. The hydrophilic polymer that can be used in the present invention means a polymer having a hydrophilic functional group in the structure thereof. The type of such hydrophilic functional group is not particularly limited. A polymer containing at least one type selected from among a hydroxyl group, an amino group, an amide group, a carboxylic acid group, and a quaternary amino group is preferable. A polymer having an amino group, an amide group, and a quaternary amino group is most preferable. Examples of a polymer having a hydroxyl group include polyvinyl alcohol, a polyethylene-polyvinyl alcohol copolymer, a partial hydrolysate of vinyl polyacetate, and partially substituted cellulose derivatives such as diacetyl cellulose, ethyl cellulose and carboxymethyl cellulose. In addition, natural products such as guar gum, pectin, starch, carrageenan, glucomannan or sialyllactose, or the synthetic products thereof may also be included. Of these examples, polyvinyl alcohol is preferable. Examples of a polymer having an amino group include polyvinylamine, and polyaminocaproic acid methacrylate. In addition, natural products such as chitosan or the synthetic products may also be included. Of these examples, polyvinylamine is preferable.

Examples of a polymer having an amide group include single polymers such as polyacrylamide or polyvinylpyrrolidone and copolymers consisting of such polymer and (meth)acrylate or a vinyl polymer such as vinyl acetate. In addition, natural products such as collagen, gelatin, fibroin, casein or kelatin, or the synthetic products thereof may also be included. (The amide group of the present invention may also include an amide group that constitutes a peptide bond.) Of these examples, polyacrylamide, polyvinylpyrrolidone, and gelatin are preferable.

Examples of a polymer having a polycarboxylic acid group include polyacrylic acid, carboxymethyl cellulose, and polylactic acid. In addition, natural products such as alginic acid or hyaluronic acid, and the synthetic products thereof may also be included. Of these examples, polyacrylic acid is preferable. A part of or the entire carboxylic acid group may be in an undissociated state, or it may form the salts of sodium, potassium, ammonium, and the like.

A cationic polymer is also preferably used. A quaternary ammonium salt group is obtained by adding halogenated alkyl or the like to an alkylamino group. Specific examples of a monomer that derives a constituent unit having a quaternary ammonium group include an N,N-dimethylaminoethyl (meth)acrylate methyl chloride quaternary product, an N,N-dimethylaminopropyl (meth)acrylamide methyl chloride quaternary product, and an N,N-diallylmethylamine methyl chloride quaternary product. Other examples of a cationic polymer include polydiallyldimethylammonium chloride, polyethyleneimine, a polyvinylpyridine quaternary salt, and a polymer having a quaternary phosphonium group. Moreover, other examples of such cationic polymer also include copolymers of these compounds and condensation products such as dicyandiamide with formalin or alkylenediamine with epichlorohydrin. Furthermore, a betaine polymer having such cationic group and an anionic group such as carboxylic acid, sulfonic acid or phosphonic acid may also be used.

The molecular weight of a hydrophilic polymer that can be used in the present invention may be arbitrarily determined depending on the type thereof, the purpose thereof, the kind of a supported antibody, and the like. In general, the weight-average molecular weight of such hydrophilic polymer is preferably 5,000 to 1,000,000, more preferably 10,000 to 500,000, and most preferably 30,000 to 300,000. The content of a hydrophilic group in the hydrophilic polymer of the present invention may be arbitrarily determined depending on the type thereof, the purpose thereof, the kind of a supported antibody, and the like. The content is preferably between 0.1 to 3 groups, more preferably between 0.3 to 1.5 groups, and most preferably 0.5 to 1 group, per monomer unit. A polymer used in coating may be used singly. Otherwise, several polymers may be mixed, or it may be used as a copolymer with any given monomer. The fact that the hydrophilic polymer of the present invention should be selected from the viewpoint of affmity, not only for an antibody, but also for a substrate material, is obvious to persons skilled in the art. That is to say, a preferred hydrophilic polymer differs depending on the type of a product selected as a substrate. From this viewpoint, a compound having a high affinity for a substrate may be mixed with the aforementioned hydrophilic polymer at any given ratio, and the mixture may be then used. Otherwise, a compound having a high affinity for a substrate may be copolymerized with the aforementioned hydrophilic polymer, and the mixture may be then used. The mixing ratio between the sugar chain affinity substance of the present invention and a hydrophilic polymer is 1:1 to 1:100, preferably 1:1 to 1:20, and most preferably 1:2 to 1:10.

### (4) Antibody

The antibody used for the hazardous substance removing material of the present invention is a protein, which is reactive (antigen-antibody reaction) specifically to a specific hazardous substance (antigen), has a molecule size of 7 to 8 nm, and is in a Y-shaped molecular form. In the Y-shape molecular structure of the antibody, a pair of branch portions of the antibody are called Fabs, and a stem portion thereof is called Fc, among which the Fab portions capture the hazardous substance.

The type of the aforementioned antibody corresponds to the type of the hazardous substance to be captured. Examples of the hazardous substance to be captured by the antibody include bacteria, fungi, viruses, allergens, and mycoplasmas. Specifically, the bacteria include, for example: the genus Staphylococcus (such as Staphylococcus aureus and Staphylococcus epidermidis), Micrococcus, Bacillus anthracis, Bacillus cereus, Bacillus subtilis, and Propionibacterium acnes, as gram-positive bacteria; and Pseudomonas aeruginosa, Serratia marcescens, Burkholderia cepacia, Streptococcus pneumoniae, Legionella pneumophilia, and Mycobacterium tuberculosis, as gram-negative bacteria. The fungi include, for example, Aspergillus, Penicillius, and Cladosporium. The viruses include influenza viruses, coronavirus (SARS virus), adenovirus, and rhinovirus. The allergens include pollens, mite allergens, and cat allergens.

In particular, in the present invention, an influenza antibody, which involves droplet infection and becomes a target of a hazardous substance removing filter, can be preferably used. As an antigen used in production of such influenza antibody, antigens such as type H1N1 virus antigen, type H3N2 virus antigen, and type B virus antigen, a triple antigen, and a H5 recombinant protein derived from avian influenza virus H5N1 can be used. The H5 recombinant protein kills chickens. Thus, in the case of this protein, an antibody cannot be obtained from a chicken egg. However, it is possible to immunize an ostrich with this recombinant protein.

Examples of a method for producing the aforementioned antibody include: a method in which an antigen is administered to an animal such as a goat, a horse, a sheep, and a rabbit, and a polyclonal antibody is purified from the blood thereof; a method in which splenic cells of an animal to which an antigen has been administered and cultured cancer cells are subjected to cell fusion and a monoclonal antibody is purified from a culture solution thereof or from a body fluid (such as ascites) of an animal in which the fussed cells have been implanted; a method in which an antibody is purified from a culture solution of genetically modified bacteria, plant cells, or animal cells into which an antibody-producing gene has been introduced; and a method in which an ostrich or a chicken to which an antigen has been administered is allowed to lay an immune egg, and an ostrich egg antibody or a chicken egg antibody is purified from yolk powders obtained by sterilizing and spray-drying the yolk of the immune egg. Of all the above methods, the method for obtaining the antibody from an ostrich egg or a chicken egg enables easy mass production of the antibody, reducing the cost of the hazardous substance removing material.

The antibody used for the hazardous substance removing material of the present invention is preferably an antibody produced from an ostrich or chicken egg.

As an antibody produced from an ostrich egg, that described in International Publication WO2007/026689 can be used, for example. According to a method using an ostrich egg, an antibody specific for a protein, which has been hardly produced by the conventional methods, can be easily produced. Thus, a large amount of homogenous antibody can be produced with no difference in lots. The term “ostrich”is used to mean Ayes belonging to Struthioniformes. Among other, Struthio camelus belonging to Struthionidae is preferably used. An antibody can be produced from an ostrich egg according to the method described in paragraphs [0007] to [0034] of International Publication WO2007/026689.

It is desirable that the carrier constituting the hazardous substance removing material of the present invention is subjected to antibacterial treatment such as coating of an agent containing an antibacterial agent and/or antifungal treatment such as coating of an agent containing an antifungal agent. The antibody is principally a protein, and particularly, the ostrich egg antibody is food, and the antibody may also accompany a protein other than the antibody. These proteins might serve as food for bacteria and fungi to proliferate. However, if the carrier is subjected to antibacterial and/or antifungal treatment, such multiplication of bacteria and the fungi is suppressed, so that a long-term storage becomes possible.

The antibacterial/antifungal agents include organic silicon quaternary ammonium salts, organic quaternary ammonium salts, biguanides, polyphenols, chitosan, silver-support colloidal silica, zeolite-support silvers, and the like. As to the treatment method, there are a post-treatment method in which an antibacterial/antifungal agent is immersed in or applied to the support made of a fiber, a raw thread/raw cotton improving method in which an antibacterial/antifungal agent is mixed in the step of synthesizing a fiber constituting the carrier, and the like.

Regarding methods for immobilizing the antibody to the carrier, there are: a method in which, after a carrier is subjected to silane treatment using γ-aminopropyl-triethoxysilane or the like, an aldehyde group is introduced on the surface of the carrier by glutaraldehyde or the like, to effect a covalent bond between the aldehyde group and an antibody; a method in which an untreated carrier is immersed into an aqueous solution of an antibody to cause ion boding, thereby immobilizing the antibody to the carrier; a method in which an aldehyde group is introduced to a carrier having a specific functional group to effect a covalent bond between the aldehyde group and an antibody; a method in which a carrier having a specific functional group is ion-bonded to an antibody; and a method in which a carrier is coated with a polymer having a specific functional group, followed by an introduction of an aldehyde group to effect a covalent bond between the aldehyde group and an antibody. In the present invention, antibody can be simply supported on a carrier by spraying a solution of antibody onto a carrier.

The hazardous substance removing material of the present invention can be used for a filter for an air purifier, a mask, a wipe sheet, and the like.

When the hazardous substance removing material of the present invention is used for an air purifier filter, it may be used in combination with the following conventional filters and any other conventional filters: a prefilter for removing dusts, a dust removal filter, a photocatalyst filter having deodorant effects, an antibacterial filter for removing other hazardous substances, and a VOC-absorbing filter.

### EXAMPLES

The features of the present invention are hereafter more specifically described with reference to examples and comparative examples. Materials, their quantities consumed, proportions thereof, contents of processing, processing procedures, and the like set forth in the following examples can be appropriately modified without departing from the sprit of the present invention. Accordingly, the scope of the present invention is not to be construed as being limited to the specific examples shown below.

### Example 1

### (Production of Carrier)

An acetone/water (97:3) solution containing cellulose acetate (total degree of substitution: 2.4; number average molecular weight: 30,000; manufactured by Aldrich) (25% by mass) was heated to 60° C. and then was squirted with air out of a nozzle of 0.1 mm in diameter at a spinning rate of 500 m/m for nonwoven fabric formation. Accordingly, a nonwoven fabric N-1 with a membrane thickness of 85 μm was obtained. A spinning cylinder was heated to 100° C. with a heater. The average fiber diameter was measured by SEM, and it was found to be 8 μm.

### (Production of Antibody)

0.5 mL of an antigen solution containing inactivated influenza virus (100 μg) was mixed with 0.5 mL, of a complete adjuvant. The obtained mixture was inoculated into the chest muscle of an ostrich for an initial immunization. For a second and the subsequent immunizations, 0.5 mL of the same above antigen solution was mixed with 0.5 mL of an incomplete adjuvant, and the obtained mixture was inoculated into the neck muscle of the ostrich every one week until the fourth week. Only yolk was collected from an egg laid by this ostrich, and it was then stirred. 10 mL of this yolk solution was mixed with TBS (20 m M Tris-HCL (pH 7.5), 0.15 M NaCL, 0.5% NaN), and 5 mL of 10% dextran sulfate/TBS was then added to the mixture. The thus obtained mixture was stirred for 30 minutes. 10 mL of 1 M CaCl₂/TBS was added to the reaction solution, and the obtained mixture was then stirred. The reaction solution was then left at rest for 2 hours or more. Thereafter, the resultant was centrifuged at 10,000 rpm for 30 minutes, and a supernatant was then recovered. Ammonium sulfate was added to the supernatant to a final concentration of 40%, and the mixture was then left at rest for 12 hours or more. The resultant was centrifuged at 10,000 rpm, and a precipitate was then recovered. This precipitate was resuspended in 10 mL of TBS, and it was then dialyzed against TBS.

### (Production of Filter Sample)

An aqueous solution of fucose α-1,6-N-acetylglucosamine (manufactured by Calbiochem Novabiochem Novagen) was diluted to a concentration of 100 ppm. Thereafter, 100 μL of the diluted solution was uniformly developed on a 10-cm² nonwoven fabric sample N-1, and it was then dried with air-blowing at 40° C. for 2 hours, so as to produce a substrate sample.

Subsequently, 1 mL of a coating solution prepared by diluting the aforementioned dialyzed solution with water to result in an antibody concentration of 100 ppm was uniformly developed on the aforementioned 10-cm² substrate sample, and it was then left at rest at room temperature for 1 hour. Thereafter, the resultant was dried with air-blowing at 40° C. for 2 hours, so as to produce a filter sample (this coating method is referred to as a “two-step coating method”).

On the other hand, 1 mL of a mixed solution of fucose α-1,6-N-acetylglucosamine and an antibody (which had been prepared so that each concentration had become 100 ppm) was developed on a 10-cm² nonwoven fabric sample, and it was then dried with air-blowing at 40° C. for 2 hours, so as to produce a filter sample (this coating method is referred to as a “one-step coating method”).

Moreover, filter samples were produced by the same above method with the exception that fucose α-1,6-N-acetylglucosamine was replaced with the samples shown in Table 1. Furthermore, standard products, on which only an antibody was supported, were also evaluated as comparative examples. As a result of surface observation by SEM (50,000 fold), in comparison with an unsupported filter, significant changes (fusion bonding, aggregation, etc.) were not found in all samples in terms of fiber diameter, fiber density, thickness, and pore diameter distribution. Thus, uniform filter samples could be obtained.

### (ELISA Measurement)

Each of the aforementioned filters was cut into a weight of 0.1 to 1.0 mg (10 samples for each standard), and it was then disposed on a 96-well immuno plate manufactured by Nunc. Subsequently, BlockAce (manufactured by Dainippon Pharma Co., Ltd.) was mixed with PBS(-1) at a ratio of 1:1 to prepare a blocking solution, and 200 μL of the blocking solution was added to the aforementioned filter, and it was then left at rest at 37° C. for 1 hour, so as to carry out a blocking treatment. As a washing solution, PBS(−) containing 0.05% TWEEN20 was used. Hereafter, washing operations were carried out 3 times each between individual steps. Subsequently, an influenza vaccine antigen (manufactured by the Kitazato Institute) was poured thereto, and it was then left at rest at 37° C. for 1 hour. Thereafter, a 20,000-fold diluted solution (PBS(−)) of an HRP-labeled antibody of anti-influenza virus IgG (manufactured by AbD) was poured thereto, and it was then left at rest at 37° C. for 1 hour. Thereafter, 3,3′,5,5′-tetramethylbenzidine (TMB; manufactured by Sigma) was poured thereto, and it was then left at rest for 15 minutes in a dark place. Thereafter, a termination solution (0.5 mol/L sulfuric acid) was poured thereto, and it was then stirred for 1 minute. 100 μL of the reaction solution was extracted, and it was then placed into another immuno plate. Thereafter, the absorbance at 450 nm (control: 620 nm) was measured with a Microplate Reader (manufactured by Bio-Rad Laboratories). It was confirmed that the degree of color development of an antibody-non-supported sample was significantly low, and that a blocking treatment was properly carried out. A comparison was made among the samples in terms of color density per unit weight.

(Note: Since the weight of an antibody and that of a polymer material were small enough to that of a substrate (up to ppm order), they are negligible.)
(Comparison with Chicken-Derived Antibody)

An antibody was produced by the same above method with the exception that a chicken was used instead of an ostrich as an animal to be immunized. Using this antibody, R-2 and N-1c that corresponded to R-1 and N-1b, respectively, were produced.

TABLE 1

| | | | ELISA | |
|---|---|---|---|---|
| | | | color | |
| | | | density per | |
| Sample | | Coating | mg of filter | |
| No. | Sugar chain affinity substance | method | sample | Remarks |
| R-1 | None | — | 0.65 | Comparative example |
| R-2 | None | — | 0.64 | Comparative example |
| | | | | (Chicken egg antibody |
| | | | | corresponding to R-1) |
| N-1a | Fucose | Two-step | 0.95 | The present invention |
| | α-1,6-N-acetylglucosamine | coating | | |
| N-1b | Fucose | One-step | 1.02 | The present invention |
| | α-1,6-N-acetylglucosamine | coating | | |
| N-1c | Fucose | One-step | 0.92 | The present invention |
| | α-1,6-N-acetylglucosamine | coating | | (Chicken egg antibody |
| | | | | corresponding to N-1b) |
| N-2a | Galactose | Two-step | 0.88 | The present invention |
| | β-1,6-N-acetylglucosamine | coating | | |
| N-2b | Galactose | One-step | 0.96 | The present invention |
| | β-1,6-N-acetylglucosamine | coating | | |
| N-3a | N-acetylgalactosamine | Two-step | 0.84 | The present invention |
| | α1-O-serine | coating | | |
| N-3b | N-acetylgalactosamine | One-step | 0.90 | The present invention |
| | α1-O-serine | coating | | |
| N-4a | N-acetylneuraminic acid | Two-step | 0.83 | The present invention |
| | | coating | | |
| N-4b | N-acetylneuraminic acid | One-step | 0.89 | The present invention |
| | | coating | | |
| N-5a | Mannose α1,3-mannose, | Two-step | 0.78 | The present invention |
| | α-methyl glycoside | coating | | |
| N-5b | Mannose α1,3-mannose, | One-step | 0.84 | The present invention |
| | α-methyl glycoside | coating | | |
| N-6a | Xylooligosaccharide | Two-step | 0.76 | The present invention |
| | | coating | | |
| N-6b | Xylooligosaccharide | One-step | 0.82 | The present invention |
| | | coating | | |
| N-7a | 2-AA glucose homopolymer | Two-step | 0.79 | The present invention |
| | | coating | | |
| N-7b | 2-AA glucose homopolymer | One-step | 0.87 | The present invention |
| | | coating | | |
| N-8a | Sugar chain No. 44* | Two-step | 1.10 | The present invention |
| | | coating | | |
| N-8b | Sugar chain No. 44* | One-step | 1.26 | The present invention |
| | | coating | | |

Galactose β-1,6-N-acetylglucosamine, N-acetylgalactosamine α1-O-serine, mannose α1,3-mannose, α-methyl glycoside (manufactured by Calbiochem Novabiochem Novagen)
N-acetylneuraminic acid (manufactured by MP Biomedicals)
Xylooligosaccharide (manufactured by Wako Pure Chemical Industries, Ltd.)
2-AA glucose homopolymer (manufactured by Prozyme)
Sugar chain No. 44* (manufactured by Masuda Chemical Industries Co., Ltd.)
*Sugar chain No. 44; Terminal pyridylamide (PA) core sugar chain standard sample

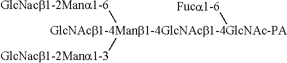

It was found that the embodiment of the present invention brings on a large amount of antigen captured per antibody, and thus that a supported antibody can be efficiently used.

### Example 2

A sample was produced and evaluated by the same above method with the exception that the hydrophilic polymers shown in Table 2 were further uniformly mixed in the one-step coating method of Example 1 using Fucose α-1,6-N-acetylglucosamine (wherein the amount of an antibody coated in the test system shown in Table 2 had previously been adjusted to become constant). Moreover, the obtained filter samples were left at rest at 60° C. at 90% RH for 1 week, and evaluation was then carried out. For comparison, a standard product (R-1) that did not contain a sugar chain affinity substance was simultaneously evaluated.

Furthermore, the average thickness of a sugar chain affinity layer was measured by the following formula obtained based on cylindrical approximation of fibers.

Average thickness=(mass of sugar chain affinity layer per unit area)/average density of composition/(2×fiber weight per unit area)/average fiber density·average fiber diameter)

TABLE 2

| | | | | ELISA color density | | |
|---|---|---|---|---|---|---|
| | | Additive | Average thickness | per mg of filter sample | | |
| | | Amount | (nm) of sugar | | After | |
| | | (mass % | chain affinity | Immediately | preservation | |
| Sample | | vs. | substance- | after | at 60° C., | |
| No. | Hydrophilic polymer | antibody) | containing layer) | production | 90% RH | Remarks |
| N-1b′ | None | — | — | 0.98 | 0.76 | The present |
| | | | | | | invention |
| N-9b | Gelatin | 10 | 1 | 1.06 | 0.90 | The present |
| | | | | | | invention |
| N-9c | Gelatin | 100 | 10 | 1.36 | 1.30 | The present |
| | | | | | | invention |
| N-9d | Gelatin | 1000 | 104 | 1.08 | 1.06 | The present |
| | | | | | | invention |
| N-10b | Polyvinylamine | 100 | 10 | 1.52 | 1.48 | The present |
| | | | | | | invention |
| N-13b | Polyacrylamide | 100 | 10 | 1.48 | 1.44 | The present |
| | | | | | | invention |
| N-14b | Polyvinylpyrrolidone | 100 | 10 | 1.62 | 1.57 | The present |
| | | | | | | invention |
| N-15b | Poly(trimethylaminoethyl | 100 | 10 | 1.55 | 1.53 | The present |
| | acrylate)chloride | | | | | invention |
| R-1′ | None (no sugar | — | — | 0.64 | 0.22 | Comparative |
| | chains) | | | | | example |

From the results shown in Table 2, it is found that (1) antibody use efficiency is increased with the use of a sugar chain affinity substance, (2) apparent antibody titer is further improved by the combined use with a hydrophilic polymer, and (3) preservative quality is significantly improved by the combined use with a hydrophilic polymer.

1. A hazardous substance removing material consisting of a carrier on which an antibody and a sugar chain affinity substance having an affinity for a sugar chain in the Fc region of the antibody are supported.

2. The hazardous substance removing material according to claim 1, wherein the antibody is IgG.

3. The hazardous substance removing material according to claim 1, wherein the sugar chain affinity substance has the same oligosaccharide unit as that of a sugar chain in the Fc region of IgG.

4. The hazardous substance removing material according to claim 1, wherein the sugar chain affinity substance is a sugar chain containing at least one type selected from among glucose, galactose, mannose, xylose, fucose, N-acetylglucosamine, N-acetylgalactosamine, and N-acetylneuraminic acid.

5. The hazardous substance removing material according to claim 1, wherein the surface of the carrier is coated with the sugar chain affinity substance.

6. The hazardous substance removing material according to claim 1, which has a hydrophilic polymer as well as the sugar chain affinity substance on the carrier.

7. The hazardous substance removing material according to claim 6, wherein the hydrophilic polymer has at least one type of functional group selected from among a hydroxyl group, an amino group, an amide group, a carboxylic acid group, and a quaternary amino group.

8. The hazardous substance removing material according to claim 1, wherein a layer comprising the sugar chain affinity substance has an average thickness of 5 to 20 nm.

9. The hazardous substance removing material according to claim 1, wherein the antibody is derived from ostriches.

10. A method for removing a hazardous substance, comprising: removing a hazardous substance from a gas phase or a liquid phase using a hazardous substance removing material consisting of a carrier on which an antibody and a sugar chain affinity substance having an affinity for a sugar chain in the Fc region of the antibody are supported.
